(19) United States
(12) Patent Application Publication
(10) Pub. No.: US 2022/0096094 A1
(43) Pub. Date: Mar. 31, 2022

(54) EXPANDABLE GUIDE DEVICES, SYSTEMS, AND METHODS

(71) Applicant: Boston Scientific Scimed, Inc., Maple Grove, MN (US)

(72) Inventors: Louis McNern, Donegal (IE); Gary Gilmartin, Foxford (IE); Damien V. Nolan, Galway (IE)

(21) Appl. No.: 17/476,960

(22) Filed: Sep. 16, 2021

Related U.S. Application Data
(60) Provisional application No. 63/083,960, filed on Sep. 27, 2020.

(51) Int. Cl. A61B 17/12; A61B 1/31; A61B 1/01

(52) U.S. Cl. CPC A61B 17/12168; A61B 1/01; A61B 1/31; A61B 17/12099

(57) ABSTRACT

The present disclosure, in its various aspects, is directed to expandable guide devices, implementation methods, and related delivery systems. Embodiments according to the present disclosure, including as described herein, may increase the effectiveness and efficiency of endoscopy procedures. In one example, an embodiment includes an expandable guide device, the device configured to slidingly receive an instrument through an instrument lumen of the device. An internal expandable member is disposed within the body and expandable radially from the inward-facing surface of the body, and an external expandable member is disposed about the body and expandable radially from the outward-facing surface. In some embodiments, the external and internal expandable members are balloons and comprise a compliant and a non-compliant material, respectively.

### PRIORITY

This application claims the benefit of priority under 35 USC § 119 to U.S. Provisional Patent Application Ser. No. 63/083,960, filed Sep. 27, 2020, which is incorporated by reference herein in its entirety and for all purposes.

### FIELD

The present disclosure pertains to medical devices. More particularly, the present disclosure pertains to expandable guide devices and related systems and methods, particularly as may increase the effectiveness and efficiency of endoscopic procedures.

### BACKGROUND

A colonoscope is a type of endoscope used for colonoscopies. Due to the length of the colonoscope and the anatomy of the colon, during some procedures, for example during polyp removal, the colonoscope can become deformed, or “loop” within the colon, possibly causing pain for the patient and/or prolonging the procedure time. Loops or angulation can result in loss of control of the colonoscope and increase discomfort for the patient. When looping occurs, the potential movements of the endoscopist may become limited. Consequently, it may become necessary to restart the colonoscopy procedure and the effectiveness and efficiency of the procedure may be compromised. Although physicians are trained in methods to reduce the prevalence of looping, it remains a recurring issue.

It is with the above considerations in mind that the improvements of the present disclosure may be useful.

### SUMMARY

The present disclosure, in its various aspects, is directed generally to expandable guide devices, implementation methods, and related systems. Embodiments according to the present disclosure, including those described herein, may increase particularly the effectiveness and efficiency of procedures used for the examination and treatment of conditions within the body, e.g., colonoscopies.

In an aspect, embodiments of the disclosure describe an expandable guide device that may comprise a body having a proximal end, a distal end, an inward-facing surface defining an instrument lumen extending therethrough along a longitudinal axis, and an outward-facing surface, the instrument lumen configured to slidingly receive a length of an instrument extendible therethrough. An internal expandable member may be disposed about the body and expandable radially from the inward-facing surface. An external expandable member may be disposed about the body and expandable radially from the outward-facing surface. An inflation fluid delivery member may be in fluid communication with each expandable member.

In various embodiments described herein and otherwise within the scope of the disclosure, the internal and external expandable members may be configured to expand independently of each other. The external expandable member may be adhered to the outward-facing surface of the body. The internal expandable member may be adhered to the inward-facing surface of the body. The external expandable member may be a balloon and may comprise a compliant material. The internal expandable member may be a balloon and may comprise a non-compliant material. The device may comprise a fixing element extendable from the distal end of the body and may be configured to assist with stabilizing the device with respect to the body lumen. The body, instrument, or both, may be adjustable in relative position with respect to each other along the longitudinal axis of the body and the length of the instrument. The fixing element may comprise at least two antennae-like structures extending distally and projecting radially from the longitudinal axis. The external expandable member may comprise a mesh-like structure. The external expandable member may have an unexpanded configuration, a partially expanded configuration, and an expanded configuration. The internal expandable member may be configured to be unexpanded, partially expanded, or expanded. The device may comprise a pressure sensor within the internal expandable member, external expandable member, or both. A distal end of the external expandable member may be tapered with a distally decreasing diameter. The internal expandable member, external expandable member, or both, may comprise a spherical-like shape, a toroidal-like shape, or an ellipsoidal-like shape, when expanded. The instrument lumen may be configured to frictionally contact the instrument when the internal expandable member is in an expanded configuration.

In an aspect, embodiments of the disclosure describe a system that may comprise an instrument and a plurality of expandable guide devices slidably disposable about the instrument, each expandable guide device comprising a body having a proximal end, a distal end, an inward-facing surface defining an instrument lumen extending therethrough along a longitudinal axis, and an outward-facing surface, the instrument lumen configured to slidingly receive a length of the instrument extendible therethrough. Each body may comprise at least one expandable member disposed about the body, and the expandable member may comprise a mesh-like structure.

In various embodiments described herein and otherwise within the scope of the disclosure, a system may further comprise a sheath disposable about the at least one expandable member of each expandable guide device.

In an aspect, embodiments of the disclosure describe a method of performing a procedure that may comprise coupling at least one expandable guide device with an instrument, each of the at least one expandable guide devices including an internal expandable member and an external expandable member disposed about a body of the expandable guide device. The method may comprise inserting the instrument and the at least one expandable guide device into a body lumen, advancing the instrument and the at least one expandable guide device toward a treatment site, and expanding one or both of the internal expandable member and external expandable member of each of the at least one expandable guide device.

In various embodiments described herein and otherwise within the scope of the disclosure, each internal expandable member may be expandable to compress the expandable guide device against the instrument. The method may further comprise expanding each external expandable member to anchor the instrument within the body lumen.

### BRIEF DESCRIPTION OF THE DRAWINGS

Non-limiting examples of the present disclosure are described by way of example with reference to the accompanying figures, which are schematic and not intended to be drawn to scale. In the figures, each identical or nearly identical component illustrated is typically represented by a single numeral. For purposes of clarity, not every component is labeled in every figure, nor is every component of each embodiment of the disclosure shown where illustration is not necessary to allow those of skill in the art to understand the disclosure. In the figures:

FIG. 1A illustrates an exemplary instrument of an embodiment of the present disclosure.

FIG. 1B illustrates a digestive tract within a body.

FIG. 1C illustrates a perspective view of an expandable guide device, according to embodiments of the present disclosure.

FIG. 1D illustrates a system with an expandable guide device in an expanded configuration within a digestive system and an exemplary instrument, such as the instrument of FIG. 1A, extended therethrough, in accordance with an embodiment of the present disclosure.

FIGS. 2A-C illustrate axial views of an expandable guide device in various configurations of expansion, according to embodiments of the present disclosure.

FIG. 3 illustrates a partial cross-sectional view of a system within a body lumen, according to embodiments of the present disclosure.

FIGS. 4A-C illustrate a partial cross-sectional view of a system within a contorted body lumen, according to embodiments of the present disclosure.

FIG. 5A-B illustrate a partial cross-sectional view of a system within a contorted body lumen, according to embodiments of the present disclosure.

FIG. 6A-C illustrate a partial cross-sectional view of a system within a body lumen, according to embodiments of the present disclosure.

It is noted that the drawings are intended to depict only typical or exemplary embodiments of the disclosure. Accordingly, the drawings should not be considered as limiting the scope of the disclosure. The disclosure will now be described in greater detail with reference to the accompanying drawings.

### DETAILED DESCRIPTION

It is the case, generally, that with any form of endoscopy or other procedure where any other instrument is inserted in the body it may be important to be able to stabilize the instrument in place once it is navigated to a desired position, to locate the instrument in a particular radial position defined by a cross-section of the body lumen, to orient an instrument in a particular manner dictated by a specific procedure, and the like.

Various embodiments according to the present disclosure are described below. As used herein, “proximal end” refers to the end of a device that lies closest to the medical professional along the device when introducing the device into a patient, and “distal end” refers to the end of a device or object that lies furthest from the medical professional along the device during implantation, positioning, or delivery.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used in connection with other embodiments whether or not explicitly described unless clearly stated to the contrary.

The detailed description should be read with reference to the drawings, which are not necessarily to scale, depict illustrative embodiments and are not intended to limit the scope of the invention.

Embodiments of the present disclosure may include an expandable guide as a device for use with an instrument. An instrument may be an endoscope such as a colonoscope, bronchoscope, duodenoscope, ureteroscope, gastroscope, catheter, tubing or the like. The device may include various components and configurations. Embodiments of this disclosure may comprise a medical device system. A medical device system may include an instrument, an expandable guide device, a proximal handle to, e.g., actuate the device, which extends to the expandable guide device, and/or a source of inflation fluid, or the like. Embodiments of the devices and systems may be used to substantially fill a body lumen around the instrument, during an endoscopy, to enable the operator to stabilize the instrument within the body, etc. Various embodiments described herein comprise an expandable guide device having at least one expandable member about an instrument lumen that can slidingly receive an instrument. The expandable member once expanded within the body lumen may inhibit or reduce movement of the instrument within the body lumen. In some embodiments, the inflation fluid may be a gas, a liquid, or both. In some embodiments, the body lumen may include a lumen, organ, vessel, passage, or the like, within, e.g., the pulmonary and/or gastrointestinal system, or the like.

Colonoscopies are a type of endoscopic procedure used to examine and treat the colon using an instrument (e.g., a colonoscope). The colonoscope, as an instrument with which an expandable guide device may be useful, is introduced into the patient via the rectum, and through the colon. The instrument may be used to take samples and/or remove polyps from the intestines during the examination. Due to the tortuous anatomy of the colon, when attempting to navigate the colonoscope through the curves of the colon, looping of the colonoscope, and consequently looping of intestines can occur during the procedure, which may cause pain for the patient and may slow the procedure. Looping may cause the instrument to become undesirably positioned within the body lumen and require additional steps to be taken by the physician. The loop may be an alpha loop, a reverse alpha loop, an N loop, a reverse splenic flexure, a gamma loop, or a reverse sigmoid spiral. A current method of resolving these loops involves proximal translation or removal of part or all of the instrument and restarting or continuing the procedure from the point at which the loop is no longer present, and the anatomy is substantially returned to a typical configuration. This process may cause discomfort and/or prolong the procedure for the patient.

A number of medical procedures, including intravascular procedures, procedures along the digestive, urinary, respiratory, reproductive and/or biliary tracts, thoracic and pulmonary procedures, etc., utilize instruments, such as endoscopes, to access tissue intended for diagnosis or treatment (e.g., “target tissue”) within the body. In some instances, an endoscope may incorporate features which assist the physician in visualizing and performing a treatment on the tissue. For example, some endoscopes may include a light and/or camera designed to illuminate and/or visualize the body lumen as the endoscope is navigated and positioned adjacent to a target tissue site. Additionally, some endoscopes may also include a lumen (e.g., a working channel) through which a resecting device, grasping member, or other accessory devices may be deployed and utilized. Additional visualization and/or external and/or internal imaging methods may be alternatively or additionally employed, e.g., fluoroscopy.

For various uses of colonoscopes or other instruments, within various body lumens and for various purposes, such as described above, embodiments of the expandable guide devices, systems and methods of the present disclosure may be utilized to enable the physician to perform procedures with greater precision, accuracy, and ease than without the expandable guide devices.

Exemplary devices, systems, and methods with which embodiments of the present disclosure may be implemented include, but are not limited to, those described in the complete disclosures of U.S. Provisional Patent Application having Attorney Docket No. 8150.0728Z, titled Devices, Systems, and Methods for Pyloric Occlusion, filed Aug. 27, 2020; U.S. Provisional Patent Application having Attorney Docket No. 8150.0729Z, titled Stabilization and Leverage Devices, Systems, and Methods, filed Aug. 28, 2020; U.S. Provisional Patent Application having Attorney Docket No. 8150.0723Z, titled Expandable Guide Devices, Systems, and Methods, filed even date herewith; and U.S. Provisional Patent Application having Attorney Docket No. 8150.0727Z, titled Expandable Guide Devices, Systems, and Methods, filed even date herewith, each of which disclosures are herein incorporated by reference in their entirety.

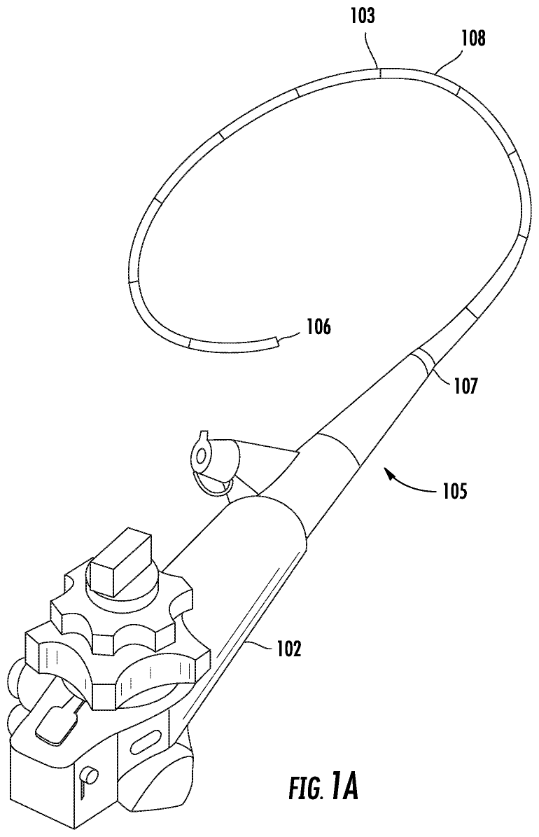

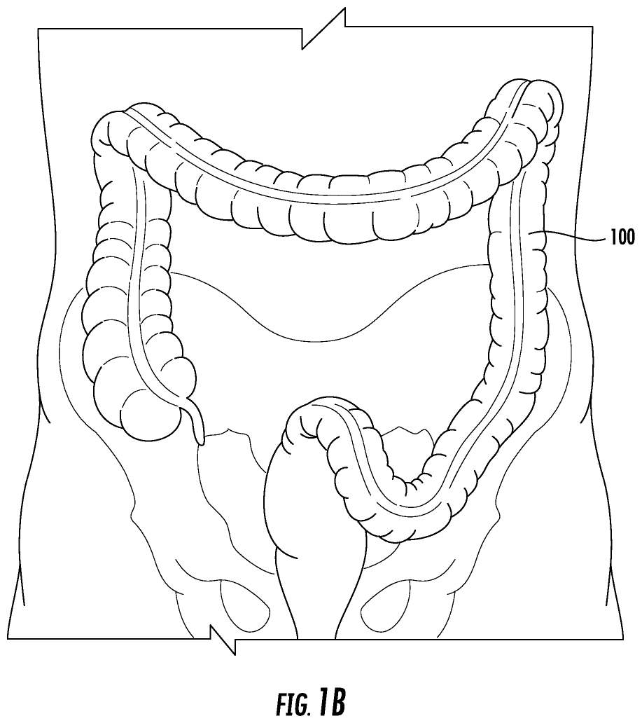

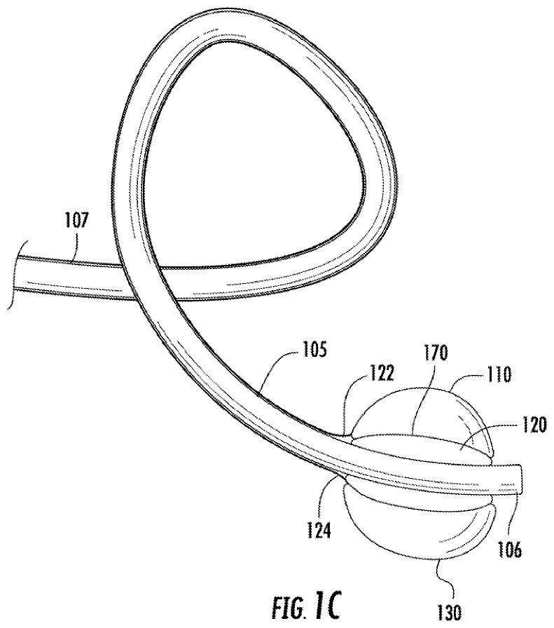

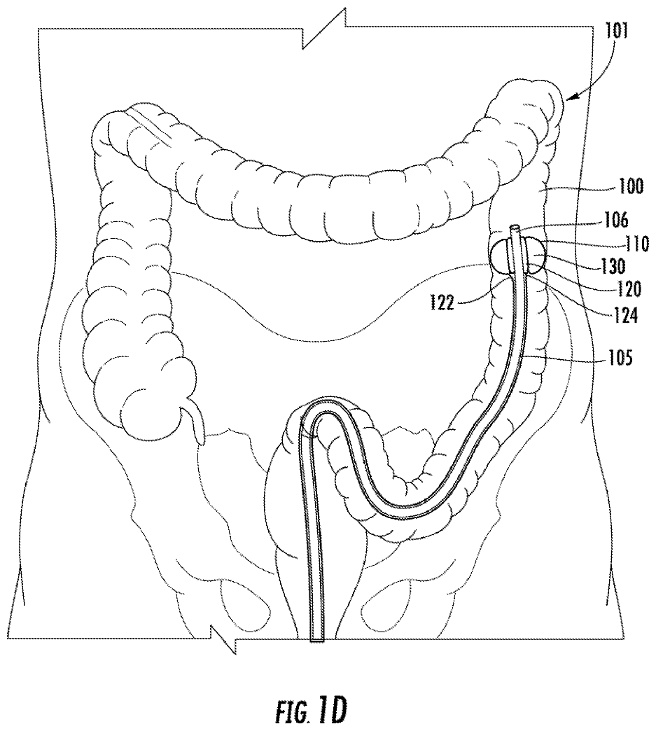

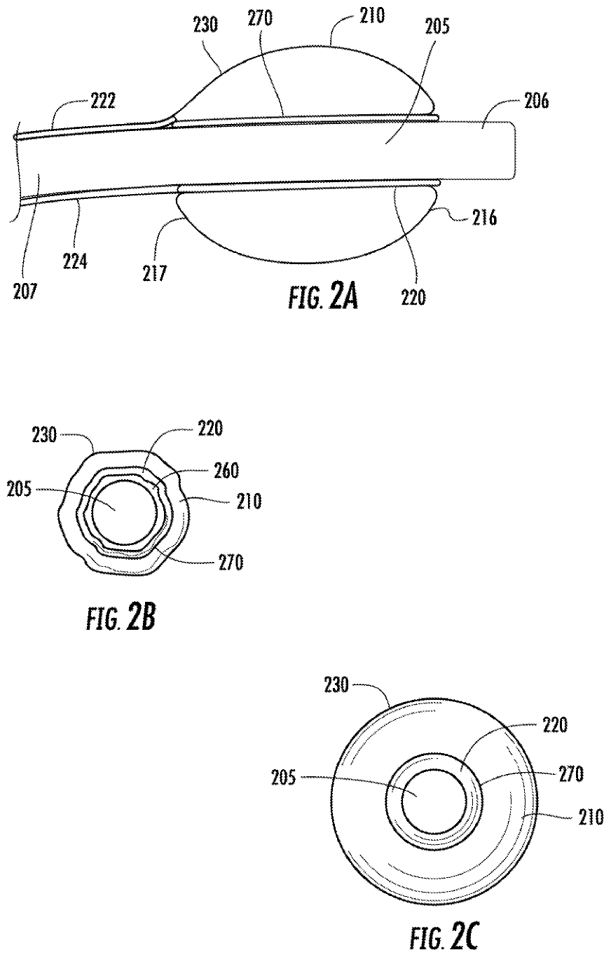

Referring to FIG. 1A, an embodiment of an instrument 105 that may be used with an expandable guide device as disclosed herein is illustrated. The instrument 105, e.g., endoscope, comprises a distal end 106 and a proximal end 107 with a lumen or working channel extending therethrough. A handle 102 at the proximal end 107 may be operated by a medical professional to manipulate the instrument 105. The instrument 105 may include cuts or channels 103 along a wall 108 of the insertion portion (e.g., flexible tubing) of the instrument 105 in order to facilitate movement and flexibility of the instrument 105 within a patient, e.g., by operation of steering knobs at the handle 102.

FIG. 1B illustrates one example of a body lumen which may be investigated by a medical device with an expandable guide device, as disclosed herein. For exemplary purposes, the body lumen 100 is the large intestine, including the colon. As may be seen, the colon presents twists and curves along its length that create challenging anatomy to navigator by simply steering the instrument from outside the body.

FIG. 1C illustrates an embodiment of a distal end of a system according to an embodiment of the present disclosure, which includes an expandable guide device 130, including an external expandable member 110 and an internal expandable member 120 with a body 170 disposed therebetween. The internal expandable member 120 forms an instrument lumen and is slidably disposed about an instrument 105 (e.g., the endoscope of FIG. 1A). FIG. 1C is a cross-section, and it should be understood that the external expandable member 110, the internal expandable member 120, and the body 170 each circumferentially surround the instrument 105. The external expandable member 110 is adhered to an outward-facing surface of the body 170. The internal expandable member 120 is adhered to an inward-facing surface of the body 170. The internal expandable member 120 defines an instrument lumen that slidingly receives a length of the instrument 105 extendible therethrough. The external expandable member 110 is in fluid communication with a first inflation fluid delivery member 122. The internal expandable member 120 is in fluid communication with a second inflation fluid delivery member 124. In various embodiments herein, one or more inflation fluid delivery members may be clipped to or otherwise extend along an instrument or along a sheath disposed about an instrument. FIG. 1C depicts both the external expandable member 110 and the internal expandable member 120 in an expanded configuration. The expansion of each expandable member may be independent, due to the independent inflation fluid delivery members 122, 124, or the expandable members 110, 120 may be simultaneous operated, e.g., by the fluid delivery members 122, 124 being supplied together with inflation fluid. The inflation fluid delivery members 122, 124 extend along the instrument 105 towards the proximal end of the instrument 105 and may connect to a handle (not shown) of the expandable guide device 130. The body 170 has a proximal end, distal end, and extends along the guide device 130 between the internal 120 and external 110 expandable members. The internal expandable member 120 is expandable radially from the inward-facing surface of the body 170. The external expandable member 110 is expandable radially from the outward-facing surface of the body 170. In the configuration of FIG. 1C, the instrument 105 is not axially extendible through the instrument lumen of the device 130 because the internal expandable member 120 is in an expanded configuration that constrains the instrument 105 from longitudinal movement with respect to the device. Therefore, with the internal expandable member 120 in the expanded configuration, the device 130 and the instrument 105 may navigate anatomy together.

FIG. 1D illustrates an embodiment of a system, e.g., the system of FIG. 1C, within the body lumen 100 (e.g., the colon). The expandable guide device 130 includes expandable members 110 and 120 that are slidably disposed about the instrument 105 (e.g., the endoscope of FIG. 1A, a colonoscope, etc.). The distal end 106 of the instrument 105 and expandable guide device 130 are advanced distally through the first portion 100 of the body lumen (i.e., the colon), with the proximal end of the instrument 105 remaining outside the body. The configuration of FIG. 1D depicts both the external expandable member 110 and the internal expandable member 120 in the expanded configuration, the expandable members 110, 120 are expanded by the inflation fluid delivery members 122, 124. Because, the instrument 105 is distally approaching a curve 101 in the body lumen 100, the expanded external expandable member 110 and internal expandable member 120 constrain the instrument 105, such that when the instrument 105 is advanced axially, the expanded expandable members assist the instrument 105 in navigating the curve 101 in the body lumen. For example, axial translation of the instrument 105 within the device 130 with the expandable members 110, 120 expanded creates a larger radius in which the instrument 105 may make the turn at the curve 101 compared to a smaller turning radius with the expandable members 110, 120 in an unexpanded configuration.

In various embodiments described herein and otherwise within the scope of the disclosure, frictional contact may be maintained between an internal expandable member and an instrument when the internal expandable member is expanded, allowing the device to move along with the instrument as the instrument is pushed or pulled through a body lumen. A stylet may also extend from a handle portion of the guide device, e.g., kept at the proximal end of the instrument and, e.g., through an actuating member of the device. The stylet can be used to push and/or pull the device in tandem with the instrument or relative to the instrument in order to position or reposition the device along the surface of the instrument. In order to move the device with the stylet, the internal expandable member is firstly unexpanded. The stylet may hold the device in place while the instrument is moved relative to the device, or vice versa. When moving the device with the stylet relative to the instrument or moving the device and the instrument in tandem, the external expandable member can be in an expanded configuration. This forces movement of a body lumen in the direction of the movement of the device and/or instrument, e.g., for navigating a looped portion of a body lumen (e.g., a colon) or unfolding tissue folds to improve visualization. The movement of the device along the instrument can be in a distal or proximal direction. Alternatively, when the external expandable member is expanded and the internal expandable member is not expanded, the stylet may be used to keep the device in place, allowing the instrument to be moved through the body lumen while the expanded external expandable member keeps the body lumen stabilized. An external expandable member may be expanded in order to contact the body lumen. An external expandable member may be partially expanded or unexpanded when moving the device through the body lumen. An internal expandable member may be expanded when moving the device with the instrument. An internal expandable member may be partially expanded or unexpanded when moving the instrument or device separately.

An expandable guide device may include one or more expandable members that may be an internal expandable member and/or an external expandable member. The expandable members may be segmented or chambered and each segment or chamber may be independently expandable, e.g., to affect different shapes of the expandable member around the circumference of the device by maintaining unexpanded, or partially expanding, or expanding, various of the chambers of the expandable member. An external expandable member may be adhered along the length of an internal expandable member. An expandable member (such as a balloon) may comprise a compliant, or elastic, material such as latex, silicone, silicone urethane co-polymers, polyethylene terephthalate (PET), nylon, polyester, Pebax, polyurethane, polyethylene, a combination thereof, or the like. Additionally, or alternatively, an external expandable may comprise an expandable mesh or mesh-like structure. In some embodiments, the internal and/or external expandable member (e.g., balloon) may be formed of a substantially non-compliant material. An expandable member may be mechanically actuated, electrically actuated, pneumatically actuated, inflated, or the like. An expandable member may be transitioned from an unexpanded configuration to an expanded configuration (including a partially and fully expanded configuration) to occlude, stretch, establish patency, or maintain patency of a body lumen. An expandable member may be used to manipulate tissue of a body lumen for viewing and/or diagnostic and/or therapeutic operations. An expandable member may be used to center or position the instrument within the body lumen. An expandable member may be a spherical-like, oblong-like, toroidal-like or ellipsoidal-like shape, when expanded. The distal end of the external expandable member may be tapered with a distally decreasing diameter in order, for example, to present a more atraumatic surface to the body lumen when advancing the device and/or instrument distally. An embodiment may include multiple expandable members around a single body. In an embodiment with multiple expandable members, each member may be independently expanded. In various embodiments, the expandable guide device may be disposable. In alternate embodiments, the expandable guide device may be reusable. In various embodiments, an expandable member may be expandable to a greater or lesser degree than a different expandable member based on, for example, different expandable volume of the expandable members, or inflating with a different volume of inflation fluid, or the like.

An expandable member or other portions of the device may comprise one or more radiopaque markers at various positions about and along the device. A radiopaque marker may be imaged during a procedure allowing for, e.g., the position of the device or devices with respect to each other, with respect to the instrument, and/or with respect to a portion of the body lumen, to be determined, changed, confirmed, etc., and the like. The degree of expansion of the expandable members may be confirmed as well. Also, contrast fluid that is imageable may be injected in the body lumen or the inflation fluid for the expandable members may be made radiopaque. Radiopaque markers may include radiopaque filler compounded into the material of the expandable member and/or independent markers may be affixed to the interior or exterior of the expandable members, to the surfaces of the body, or to other portions of the device.

An expandable guide device may have one or more inflation fluid delivery members that may extend along the instrument and/or along or through the body of an expandable guide) and through the outer surface of an expandable member. The delivery members are in fluid communication with the expandable members. The inflation fluid delivery members may supply and/or return inflation fluid for expanding and/or un-expanding or collapsing an expandable member. Each expandable member may have its own inflation fluid delivery member, or an inflation fluid deliver member may be in fluid communication with more than one expandable member. An inflation fluid delivery member may extend to the proximal end of the instrument and be connectable with a fluid source, e.g., a gas or liquid inlet/outlet at the handle of the guide device. Fluid (e.g., saline, air, etc.) may be inserted and removed from the inflation fluid delivery member by a source e.g., a syringe. The insertion of fluid may cause the expandable members to expand, and removal of fluid may cause the expandable members to un-expand, or deflate in the case, e.g., of a balloon as an expandable member. An inflation fluid delivery member may extend along the outer surface of an instrument or within a working channel or inflation lumen of an instrument. An inflation fluid delivery member may be in fluid communication with an inflation lumen of an instrument, while the instrument is extended within an instrument lumen of an expandable guide device.

FIG. 2A illustrates a view of a distal end of a system according to an embodiment of the present disclosure. An expandable guide device 230 is slidably disposed about an instrument 205 (e.g., the endoscope of FIG. 1A, a colonoscope, etc.). The device 230 is about the distal end 206 of the instrument 205. The expandable guide device 230 comprises an external expandable member 210, a body 270, an internal expandable member 220, an external inflation fluid delivery member 222 extending to the external expandable member 210, and an internal inflation fluid delivery member 224 extending to the internal expandable member 220. FIG. 2A is a cross-section, and it should be understood that the external expandable member 210, the internal expandable member 220, and the body 270 each circumferentially surround the instrument 205. The external and internal expandable members 210, 220 are distal to the fluid delivery members 222, 224, with the fluid delivery members 222, 224 in fluid communication with the expandable members 210, 220 at the proximal end 217 of the expandable members 210, 220. The fluid delivery members 222, 224 extend along the instrument 207 toward the proximal end of the instrument 207. Alternatively, the body 270 of the guide device may extend (e.g., as a sheath) about the length of the instrument 205 to a proximal end that may be coextensive with a proximal end of the instrument 205. The body 270 may transition at its proximal end to a handle, which may be used to actuate the expandable members and/or change the position of the guide device 230 relative to the instrument 205 and/or a body lumen. In such a case, the fluid delivery members 222, 224 may be embedded within the body 270 along the length of the instrument 205 or they may extend along an external or internal surface of the body 270. A distal end 216 of the expandable members 210, 220 are oriented towards the distal end 206 of the instrument 205. The device of FIG. 2A depicts a partially expanded external expandable member 210 and an unexpanded internal expandable member 220. The unexpanded internal expandable member 220 allows the user to move the device axially and rotationally along and about the instrument 205, and the partially expanded external expandable member 210 facilitates movement of the device 230 within a body lumen. The external expandable member 210 is a compliant expandable member, so as to expand such that it may fully contact, and in some cases dilate, a body lumen, pressing against and conforming to body lumen walls. The internal expandable member 220 is a non-compliant expandable member, so that it may grip the instrument 205 when expanded and may not extend outwardly into the body 270 and/or the external expandable member 210.

FIGS. 2B and 2C illustrate axial views of the system of FIG. 2A in various configurations of the instrument and guide device. FIG. 2B depicts the expandable guide device 230 in an unexpanded configuration, while FIG. 2C depicts the device 230 in an expanded configuration. In FIG. 2B, neither the external 210 nor internal 220 expandable members are expanded about the instrument 205, such that the instrument 205 is moveable freely within an instrument lumen 260 of the internal expandable member 270 and the device 230 is moveable with respect to a body lumen. The external expandable member 210 and internal expandable member 220 are disposed on opposing sides of the body 270. In FIG. 2C, both the external 210 and internal 220 expandable members are expanded about the instrument 205. When a user requires the use of the expandable guide device 230, at least one of the expandable members 210, 220 may be at least partially expanded. The expandable members 210, 220 may be independently transitioned from the unexpanded configuration of FIG. 2B to the expanded configuration of FIG. 2C, e.g., by supplying an inflation fluid through the inflation fluid delivery members 222, 224 in fluid communication with the expandable members 210, 220, e.g., as described herein. The external expandable member 210 may be inflated such that it contacts a wall of a body lumen. As the external expandable member 210 contacts the wall of the body lumen, the instrument 205 may be radially moved away from the wall of the body lumen, e.g., centering the device 205 in the body lumen or locating the device 205 in a desired radial position with respect to a radial cross section of the body lumen. The external expandable member 210 may substantially fill a section of the body lumen while in the expanded configuration, e.g., establishing patency. As the external expandable member 210 makes substantial contact with the wall of the body lumen, the device 230 may be substantially anchored within the body lumen. The body lumen (e.g., colon) may be substantially occluded by the external expandable member 210 when the external expandable member 210 is in an expanded state. The internal expandable member 220 may be inflated such that it radially compresses along the instrument 205. As the internal expandable member 220 contacts the instrument 205, it restricts axial movement of the instrument 205 relative to the instrument lumen 260 of the expandable guide device 230. Movement of the instrument 205 and the expandable guide device 230 may be manipulated in tandem with the internal expandable member 220 in an expanded state.

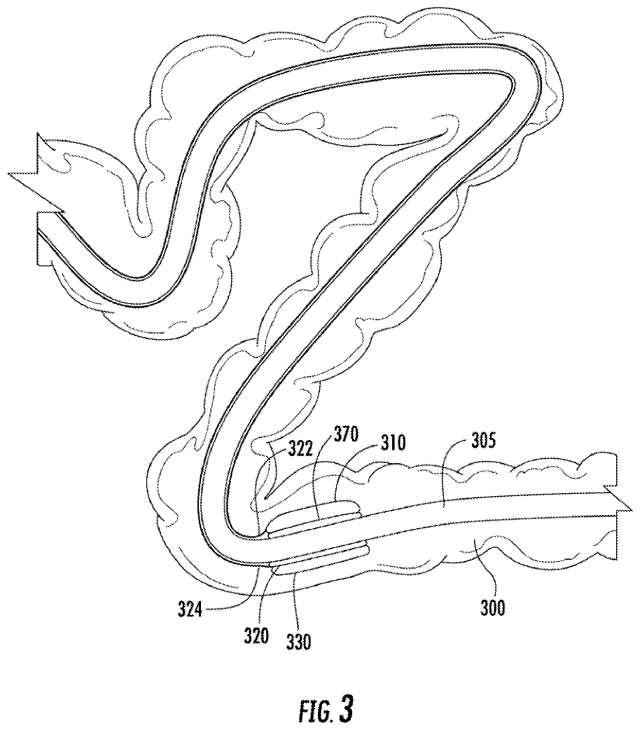

FIG. 3 depicts a view of system within a body lumen 300 according to an embodiment of the present disclosure. An expandable guide device 330 includes an external expandable member 310 and an internal expandable member 320 with a body 370 disposed therebetween. The internal expandable member 320 forms an instrument lumen and is slidably disposed about an instrument 305 (e.g., an endoscope). FIG. 3 is a cross-section, and it should be understood that the external expandable member 310, the internal expandable member 320, and the body 370 each circumferentially surround the instrument 305. The device 330 is slidably disposed about an instrument 305 within the body lumen 300 for instrument manipulation while examining the body lumen 300. The external expandable member 310 is in fluid communication with an inflation fluid delivery member 322. The internal expandable member 320 is in fluid communication with an inflation fluid delivery member 324. The expansion of each expandable member 310, 320 may be independent, due to the independent inflation fluid delivery members 322, 324. The inflation fluid delivery members 322, 324 may extend along the instrument 305 towards the proximal end of the instrument 305 and may connect to a handle (not shown) of the expandable guide device 330. The body lumen 300 is in a desired position for further navigation of the instrument 305 (e.g., is not tangled or folded and is generally patent) and therefore the external expandable member 310 is partially expanded, facilitating movement of the instrument 305 and the device 330 through the body lumen 300 while maintaining frictional contact between the internal expandable member 320 and the instrument 305 with the internal expandable member 320 in an expanded configuration.

In various embodiments described herein and otherwise within the scope of the disclosure, the body, instrument, or both, are adjustable in relative position with respect to each other along the longitudinal axis of the body and the length of the instrument. The body of the device may be moved with respect to the instrument while the internal expandable member is unexpanded regardless of whether the external expandable member is expanded or unexpanded. The device, wherein the internal expandable device is expanded, may move through the body lumen together with the instrument as a system.

In various embodiments described herein and otherwise within the scope of the disclosure, the system may comprise multiple guide devices, each with various combinations of internal and external expandable members. For example, each device with expandable members may be moveable independently along the instrument. Alternatively, devices may be connected and moved together. A first device with expandable members may be held stationary (e.g., with a stylet, using the fluid delivery lumens as stylets, or by manipulating the proximal end of a device sleeve, or the like) while a second device with expandable member(s) may be moved proximally or distally along the instrument. Expandable members may be moved with respect to instrument, body lumen, or both while partially expanded or unexpanded. Multiple devices with expandable members may be present in a system, or a device may have multiple bodies each with expandable members, or a device may have multiple expandable members along a single body. In such embodiments, the expandable members may be expanded, partially expanded, unexpanded, or combinations thereof, and moved with respect to instrument, body lumen, or both, independently of one another, dependent on one another, or combinations thereof.

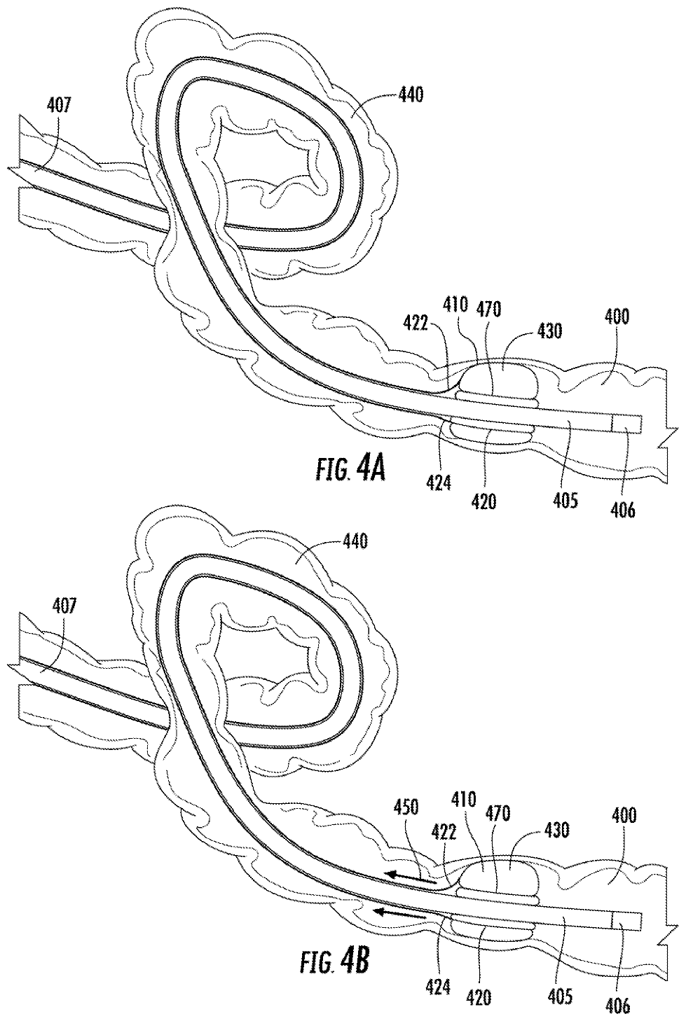

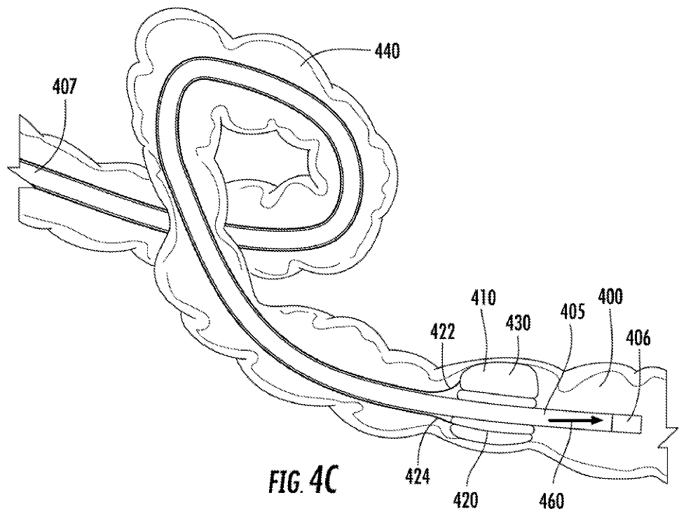

FIGS. 4A-4C depict a system within a body lumen 400 according to an embodiment of the present disclosure. An expandable guide device 430 includes an external expandable member 410 and an internal expandable member 420 with a body 470 disposed therebetween. The internal expandable member 420 forms an instrument lumen and is slidably disposed about an instrument 405 (e.g., an endoscope). FIGS. 4A-4C are a cross-sectional view, and it should be understood that the external expandable member 410, the internal expandable member 420, and the body 470 each circumferentially surround the instrument 405. The body lumen 400 has been moved into an undesirable orientation 440 (e.g., a loop) during the procedure of inserting the system into the patient. When the undesirable orientation 440 forms, the medical professional should not or cannot continue to distally translate the instrument 405 through the body lumen 400. The device 430 may be operated as described herein to resolve the loop. For example, one option to address the undesirable orientation 440 is illustrated in FIG. 4B, which shows the external expandable member 410 of the expandable guide device 430 partially expanded. As shown by force vectors 450, the internal expandable member 420 may be in the unexpanded configuration, allowing the expandable guide device 430 to be moved independent of the instrument 405 toward the proximal end 407 of the instrument 405. This proximal translation of the device 430—with the external expandable member 410 partially expanded to take up volume within the body lumen 400, while still allowing for independent rotation of the body lumen 400 with respect to the device 430 and the instrument 405—through the body lumen 400 moves (e.g., substantially reorients or substantially straightens out) the undesirable orientation 440 into a pre-procedure configuration. In various embodiments, the external expandable member 410 and the internal expandable member 420 may be unexpanded and moved proximal to the undesirable orientation portion of the body lumen 400. The external expandable member 410 may then be expanded, engaging/gripping the walls of the body lumen 400, and the device 430 may be pulled proximally. This movement may pull the body lumen 430 proximally as well, returned into a pre-procedure configuration, independent of or with further proximal movement by the instrument 405. Alternatively, the undesirable orientation 440 may be addressed as illustrated in FIG. 4C. The expandable members 410 and 420 of the expandable guide device 430 are expanded, thereby substantially locking the instrument 405 and the expandable guide device 430 with respect to each other and with respect to the body lumen 400. As shown by the force vector 460, the instrument 405 and the device 430 are moved distally, which will drag the body lumen 400 distally and allow the undesirable orientation 440 to return to the pre-procedure configuration. The external expandable member 410 can also be used to move the body lumen 400 itself with the device 430, but independent of the instrument 405. When expanded, the external expandable member 410 may grip the body lumen 400. In another embodiment, the external expandable member 410 may be expanded, gripping the body lumen, and the internal expandable member may be unexpanded, allowing movement of the instrument. While maintaining the position of the instrument, the device may be pushed distally in order to pull the body lumen out of the undesirable position. Other examples of manipulating the device and instrument with respect to each other, and/or individually or together with respect to the body lumen, are contemplated.

In various embodiments described herein and otherwise within the scope of the disclosure, the device may comprise a sheath running the length of the instrument. In such embodiments, the external expandable member may be expanded against the body lumen and the instrument pulled proximally, just proximal of the undesirable orientation. The device may then be pushed distally, pushing the body lumen out of the undesirable orientation. Alternatively, the device may be pulled proximal to the undesirable orientation and the external expandable member expanded. The instrument may then be pulled proximally, just out of the undesirable orientation, the internal expandable member expanded, and both the device and the instrument pulled proximally, removing the undesirable orientation. Alternatively, the user may pull only the device and body lumen proximally relative to the instrument, removing the undesirable orientation. In each of these embodiments, the instrument remains within the sheath, and thus the device remains disposed around the instrument and may be further reengaged with the instrument and both advanced through the body lumen.

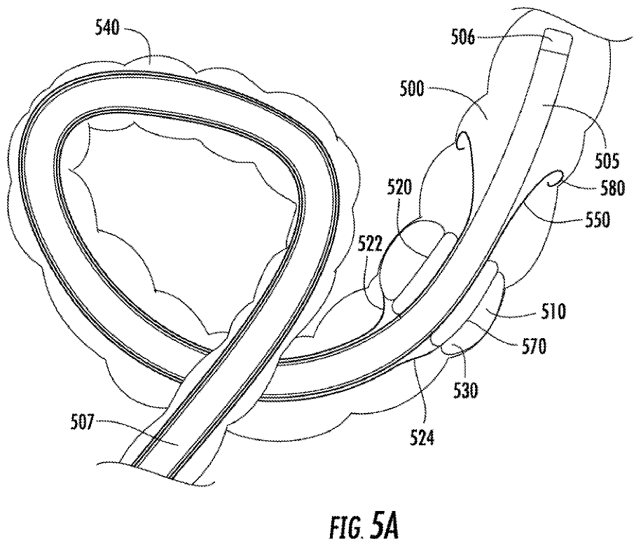

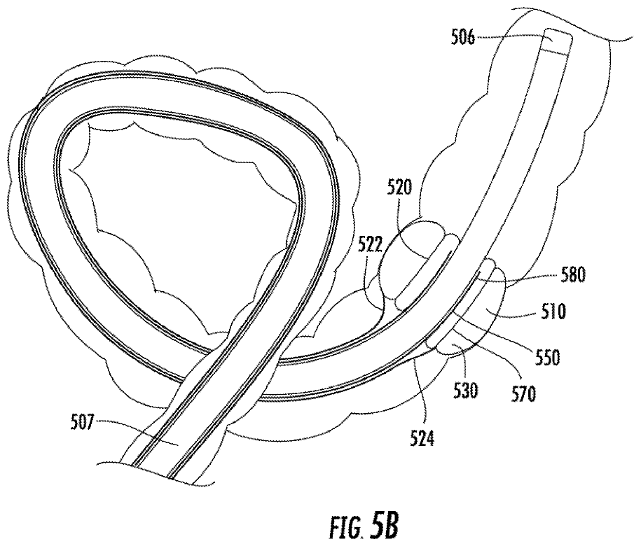

FIG. 5A depicts a view of a system within a body lumen 500 according to an embodiment of the present disclosure. The device may be operated as described above. In this view, an expandable guide device 530 is disposed about an instrument 505. Fixing element 550 extends distally along the instrument 505 and between the internal expandable member 520 and the instrument 505. The fixing element 550 projects radially from a longitudinal axis. The fixing element 550 includes distal ends 552 having a curled shape-memory such that they automatically contact the wall of the body lumen 500 in an antennae-like structure. The fixing element 550 may assist with stabilizing the device 530 with respect to the body lumen, provide substantial anchoring for axial movement of the instrument 505, and/or may assist the external expandable member 510 with moving the body lumen 500 out of the undesirable orientation 540. If an undesirable orientation of the body lumen develops, the fixing element 550 may be deployed by pushing the elements distally, which allows the fixing element 550 to extend to the body lumen wall. The ends of the fixing element 550 act as fixed points, which may assist in the elongation of the undesirable orientation by pushing or pulling the device. In various embodiments, the fixing element 550 may have a relaxed spring force as illustrated in FIG. 5A, may be heat-activated to transition into the shape illustrated in FIG. 5A, or the like. FIG. 5B illustrates the fixing element 550 in an unextended configuration internally within the internal expandable member 520 of the device 530. The fixing element 550 may be proximally retracted from the deployed configuration of FIG. 5A to the delivery configuration of FIG. 5B by proximally translating the fixing element 550 along the device 530 to substantially straighten the fixing elements.

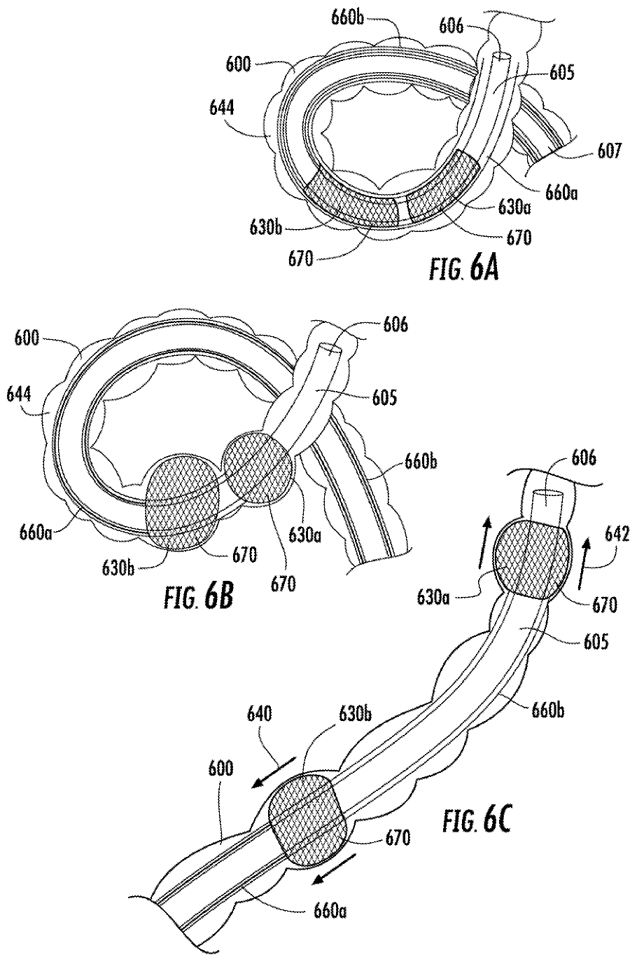

FIG. 6A-C depict views of a system within a body lumen 600 according to an embodiment of the present disclosure. Expandable members 630a and 630b are disposed about an instrument 605 toward a distal end 606 of the instrument 605. A sheath 660 is extended over the members 630a, 630b such that they are in an unexpanded configuration in FIG. 6A. In FIG. 6B, the expandable members 630a and 630b are self-expandable to an expanded configuration by proximally retracting the sheath 660. Although both expandable members 630a, 630b are expanded, they may be independently expanded by proximally translating the sheath 660 such that a distal end of the sheath 660 is between the expandable members 630a, 630b. In order to stabilize a flexible tube of an endoscope in a body lumen, or to straighten an undesired orientation 644 of the body lumen 600, one or both of the expandable members 630a, 630b in the expanded configuration may be movable along the flexible tube, towards the proximal end and distal end 606 of the flexible tube, as illustrated by the proximal force vectors 640 and the distal force vectors 642. This opposing movement of the expandable members 630a, 630b is achieved by the movement of a distal sheath 660b and a proximal sheath 660a, which are connected to the respective expandable members 630a, 630b. The congruent proximal and distal translation of the expandable members 630a, 630b may be used to manipulate the body lumen 600 out of the undesirable orientation 644. Although two expandable members 630a, 630b are illustrated, in various embodiments any number of expandable members 630a, 630b may be employed, e.g., 1, 3, 4, 5, 8, 10, 15, 20, etc. For example, if an undesirable orientation of the body lumen develops, the expandable members may be pulled proximally, such that the expandable members are within the “loop” of the undesirable orientation. The sheath may be pulled proximally, allowing the expandable members to expand. The distal expandable member 630a may then be pushed distally, and the proximal expandable member 630b pulled proximally, by their respective sheaths 660b, 660a. This movement of the expandable members may move the body lumen to a pre-procedure orientation.

In various embodiments described herein and otherwise within the scope of the disclosure, the system may comprise a sheath that extends to the proximal end of the instrument. The sheath may have graduated markings at the proximal end visible to the user outside of the body lumen during the procedure. These markings can be used by the user to track placement of the device along and with respect to the instrument and/or the body lumen. The inflation lumens may alternatively or also have graduated markings which may be used to determine similar placement of the device. The latter may be necessary in embodiments where the body of the guide device does not extend as a sheath to outside of the body lumen.

In various embodiments described herein and otherwise within the scope of the disclosure, the system may comprise a sheath. The sheath may comprise the body of the device, where the external and internal expandable members are located and may extend proximally, to the proximal end of the instrument. This sheath may be used to move a plurality of devices of a system, and/or a plurality of expandable members of a device, along the instrument.

In various embodiments, the distal end of the instrument may be axially extended distally past the distal end of the expandable guide device for navigation and viewing. An expandable guide device may be distally extended past the distal end of an instrument to manipulate a body lumen.

In various embodiments, a method of performing a colonoscopy may include coupling an expandable guide device with an instrument. The instrument and expandable guide device may be inserted together into a patient. The external expandable member can be expanded within the patient, dilating the patient's body lumen, e.g., the colon or the like. The expandable guide device increases in diameter when the external expandable member is expanded. The expansion of the expandable guide device may provide pressure against the body lumen but still allow movement of the device proximally and distally. Alternatively, expansion of the internal expandable member may inhibit movement such that the instrument and expandable guide device move together through the body lumen. The guide device may be translated through the body lumen without the instrument by use of a stylet, e.g., as described above. Expansion of the external expandable member may anchor the instrument within the body lumen.

In various embodiments, the device may comprise a pressure sensor within the internal expandable member, external expandable member, or both.

Variations, modifications, and other implementations of the present disclosure in addition to the various embodiments described herein will occur to those of ordinary skill in the art. Accordingly, the present disclosure is to be defined not by the preceding illustrative description but instead by the following claims:

What is claimed is:

1. An expandable guide device, comprising:
a body having a proximal end, a distal end, an inward-facing surface, and an outward-facing surface;
an internal expandable member disposed within the body and expandable radially from the inward-facing surface, the internal expandable member defining an instrument lumen extending therethrough along a longitudinal axis, the instrument lumen configured to slidingly receive a length of an instrument extendible therethrough;
an external expandable member disposed about the body and expandable radially from the outward-facing surface;
an internal inflation fluid delivery member in fluid communication with the internal expandable member; and
and external inflation fluid delivery member in fluid communication with the external expandable member.

2. The device of claim 1, wherein the internal and external expandable members are configured to expand independently of each other.

3. The device of claim 1, wherein the external expandable member is adhered to the outward-facing surface, and the internal expandable member is adhered to the inward-facing surface.

4. The device of claim 1, wherein the external expandable member is a balloon and comprises a compliant material.

5. The device of claim 1, wherein the internal expandable member is a balloon and comprises a non-compliant material.

6. The device of claim 1, further comprising a fixing element extendable from the distal end of the body and configured to assist with stabilizing the device with respect to the body lumen.

7. The device of claim 6, wherein the body, instrument, or both, are adjustable in relative position with respect to each other along the longitudinal axis of the body and the length of the instrument.

8. The device of claim 6, wherein the fixing element comprises at least two antennae-like structures extending distally and projecting radially from the longitudinal axis.

9. The device of claim 1, wherein the external expandable member comprises a mesh-like structure.

10. The device of claim 1, wherein the external expandable member has an unexpanded configuration, a partially expanded configuration, and an expanded configuration.

11. The device of claim 10, wherein the internal expandable member has an unexpanded configuration, a partially expanded configuration, and an expanded configuration.

12. The device of claim 1, further comprising a pressure sensor within the internal expandable member, external expandable member, or both.

13. The device of claim 1, wherein a distal end of the external expandable member is tapered with a distally decreasing diameter.

14. The device of claim 11, wherein the internal expandable member, external expandable member, or both, comprise a spherical-like, a toroidal-like, or an ellipsoidal-like shape, when in the expanded configuration.

15. The device of claim 1, wherein the internal expandable member is configured to frictionally contact the instrument when the internal expandable member is in the expanded configuration.

16. A system, comprising:
an instrument; and
at least one expandable guide device slidably disposable about the instrument, each of the at least one expandable guide device comprising a body having a proximal end, a distal end, an inward-facing surface defining an instrument lumen extending therethrough along a longitudinal axis, and an outward-facing surface, the instrument lumen configured to slidingly receive a length of the instrument extendible therethrough;
wherein each body comprises at least one expandable member disposed about the body; and
wherein the at least one expandable member comprises a mesh-like structure.

17. The system of claim 16, further comprising a sheath disposable about the at least one expandable member of each expandable guide device.

18. A method of performing a procedure, comprising:
coupling at least one expandable guide device with an instrument, each of the at least one expandable guide devices including an internal expandable member and an external expandable member disposed about a body of the expandable guide device;
inserting the instrument and the at least one expandable guide device into a body lumen;
advancing the instrument and the at least one expandable guide device toward a treatment site; and
expanding one or both of the internal expandable member and external expandable member of each of the at least one expandable guide device.

19. The method of claim 18, wherein each internal expandable member is expandable to compress the expandable guide device against the instrument.

20. The method of claim 18, further comprising expanding each external expandable member to anchor the instrument within the body lumen.
